(19) United States
(12) Patent Application Publication
(10) Pub. No.: US 2014/0142127 A1
(43) Pub. Date: May 22, 2014

(54) Stable Compositions of HMG-COA Reductase Inhibitors and Omega-3 Oils

(71) Applicant: Almburg, LLC, Wellesley, MA (US)

(72) Inventor: Orn Almarsson, Shrewsbury, MA (US)

(73) Assignee: Almburg, LLC, Wellesley, MA (US)

(21) Appl. No.: 14/060,205

(22) Filed: Oct. 22, 2013

Related U.S. Application Data
(60) Provisional application No. 61/795,706, filed on Oct. 23, 2012.

(51) Int. Cl. A61K 47/02; A61K 31/202; A61K 31/505; A61K 31/40

(52) U.S. Cl. CPC A61K 47/02; A61K 31/40; A61K 31/202; A61K 31/505
USPC 514/275; 514/423

(57) ABSTRACT

Disclosed are stable formulations of omega-3 oils and HMG-CoA reductase inhibitors. The invention comprises an omega-3 oil, a salt form of an HMG-CoA reductase inhibitor, and a salt. These compositions are stable in liquid form useful for combination therapy.

### RELATED APPLICATION

This application claims the benefit of U.S. Provisional Application No. 61/795,706, filed on Oct. 23, 2012. The entire teachings of the above application are incorporated herein by reference.

### BACKGROUND

It is desirable to have stable and bioavailable compositions of omega-3 oils and HMG-CoA reductase inhibitors to manage cardiovascular conditions. Due to the instability of HMG-CoA reductase inhibitors in liquid or semi-liquid compositions, significant difficulties exist to co-formulate omega-3 oils and HMG-CoA reductase inhibitors.

### SUMMARY

Applicants have invented stable formulations of omega-3 oils and HMG-CoA reductase inhibitors. The invention comprises an omega-3 oil, a salt form of an HMG-CoA reductase inhibitor, and a salt. These compositions are stable in liquid form useful for combination therapy. Advantageously, multiple different HMG-CoA reductase inhibitors can be stabilized in omega-3 oil with the addition of a salt. HMG-CoA reductase inhibitors have a structurally similar delta-hydroxy acid moiety, which is very sensitive to ring closure. This invention protects the HMG-CoA reductase inhibitors from degradation and allows sufficient stability for a room temperature stable oral dosage form containing an omega-3 oil and an HMG-CoA reductase inhibitor.

In one embodiment, the invention is a pharmaceutical composition comprising a capsule containing an omega-3 oil, a salt form of an HMG-CoA reductase inhibitor, and a salt, wherein the salt form of HMG-CoA reductase inhibitor and the salt contain the same counterion. In another embodiment, the invention is a capsule containing an omega-3 oil, an HMG-CoA reductase inhibitor, and 0.05 to 1.5 mmol/ml of calcium. In a further embodiment, the invention is a capsule or other oral dosage form containing an omega-3 oil, an HMG-CoA reductase inhibitor, and calcium wherein the ratio of HMG-CoA reductase inhibitor:calcium ranges from 10:0.0.05 to 10:1.5. In a still further embodiment, the invention is a dosage form comprising an omega-3 oil, an HMG-CoA reductase inhibitor, and a salt selected from calcium phosphate, calcium carbonate, and calcium chloride.

In one aspect of the invention, the HMG-CoA reductase inhibitor is selected from the list consisting of atorvastatin, rosuvastatin, pravastatin, lovastatin, pitavastatin, and simvastatin. In a further aspect of the invention, the HMG-CoA reductase inhibitor is selected from the list consisting of atorvastatin calcium, rosuvastatin calcium, pravastatin calcium, pitavastatin calcium, fluvastatin calcium. In a still further aspect of the invention, the HMG-CoA reductase inhibitor is suspended in the omega-3 oil. In another aspect of the invention, the HMG-CoA reductase inhibitor is suspended in the omega-3 oil at a concentration of 2.5-40 mg/ml. In a further aspect of the invention, a capsule contains between about 2.5 mg and about 40 mg of the HMG-CoA reductase inhibitor. In another aspect of the invention, the HMG-CoA reductase inhibitor is a salt selected from calcium sodium or potassium.

In one aspect of the invention, the omega-3 oil comprises eicosapentaenoic acid ((5Z,8Z,11Z,14Z,17Z)-eicosa-5,8,11,14,17-pentaenoic add). In another aspect of the invention, the omega-3 oil comprises docosahexaenoic acid ((4Z,7Z,10Z,13Z,16Z,19Z)-docosa-4,7,10,13,16,19-hexaenoic acid). In a further embodiment of the invention, the omega-3 oil comprises eicosapentaenoic acid and docosahexaenoic acid. In one aspect of the invention, the omega-3 oil comprises at least 75% eicosapentaenoic acid. In another aspect of the invention, the omega-3 oil comprises at least 75% docosahexaenoic acid. In a further embodiment of the invention, the omega-3 oil comprises at least 75% eicosapentaenoic acid and docosahexaenoic acid. In further embodiments, the omega-3 oil, the eicosapentaenoic acid, the docosahexaenoic acid, or the eicosapentaenoic acid and docosahexaenoic acid combined have a purity of at least 80%, at least 90% or at least 95%.

In one embodiment, the omega-3 oil is contained in a capsule. In one aspect of the invention, the capsule contains at least 80% omega-3 oil by weight (excluding the weight of the capsule). In another aspect of the invention, the capsule contains at least 80% omega-3 by weight (excluding the weight of the capsule) and the omega-3 has a purity of at least 85%.

In one aspect of the invention, the omega-3 oil is an omega-3 acid ethyl ester. In another aspect of the invention, the omega-3 oil is an omega-3 acid triglyceride. In a further aspect of the invention, the omega-3 oil is an omega-3 free fatty acid.

In one embodiment, a capsule contains an omega-3 oil, an HMG-CoA reductase inhibitor, and a salt wherein at least 10% of the HMG-CoA reductase inhibitor is in solution in the omega-3 oil. In another embodiment, a capsule contains an omega-3 oil, an HMG-CoA reductase inhibitor, and a salt wherein at least 25% of the HMG-CoA reductase inhibitor is in solution in the omega-3 oil. In one embodiment, a capsule contains an omega-3 oil, an HMG-CoA reductase inhibitor, and a salt wherein at least 50% of the HMG-CoA reductase inhibitor is in solution in the omega-3 oil.

In one embodiment, a pharmaceutical compositions comprises an omega-3 oil, a salt form of an HMG-CoA reductase inhibitor, and a salt, wherein the salt form of HMG-CoA reductase inhibitor and the salt contain the same counterion.

In one aspect of the invention, the salt is a calcium salt. In another aspect of the invention, the salt is selected from the list consisting of calcium phosphate, calcium carbonate, and calcium chloride. In another aspect of the invention, the counterion of the salt is present at a concentration of 0.05 to 1.5 mmol/ml. In a further aspect of the invention, the counterion is calcium and said calcium is present at a concentration of 0.05 to 1.5 mmol/ml. In one aspect of the invention, the dosage form contains about 0.05 to 1.5 mmol/ml of calcium.

In one embodiment, a dosage form contains between 500 and 1500 mg of omega-3 oil.

### DESCRIPTION

### Definitions

An “omega-3 oil” is any oil comprising omega-3 fatty acids, omega-3 mono-, di-, or triglycerides, phospholipids, or omega-3 free fatty acids, omega-3 esters, including, but not limited to, omega-3 alkyl esters. Omega-3 oils can be characterized using two unique descriptors, species and component. The species of an omega-3 oil is determined by the structure of the polyunsaturated carbon chain bound to the carboxyl group. The component of an omega-3 oil is determined by the chemical nature of the carboxyl group. For example, omega-3 fatty acids employ a —COOH structure bound to the polyunsaturated carbon chain, omega-3 esters employ a —COOR structure bound to the polyunsaturated carbon chain, and omega-3 mono- di- or triglycerides employ a —COOR′ structure bound to the polyunsaturated carbon chain, where R′ comprises a glycerol backbone. Several omega-3 oils which can be used in making formulations of the invention include, but are not limited to, omega-3 oils such as Omegabrite® (Omega Natural Science), Epanova™ (TillottsPharma AG), OMEGA-3/90 (K D Pharma), Epax™ (PronovaBiocare AS), Vascazen (Pivotal Therapeutics), Incromega (Croda/Bioriginal), Lovaza (Pronova), and Vascepa (Amarin).

“EPA” is defined as eicosapentaenoic acid (C20:5 omega-3 acid, chemically known as (5Z,8Z,11Z,14Z,17Z)-eicosa-5,8,11,14,17-pentaenoic acid)), and “DHA” is defined as docosahexaenoic acid (C22:6 omega-3 acid, chemically known as (4Z,7Z,10Z,13Z,16Z,19Z)-docosa-4,7,10,13,16,19-hexaenoic acid). Both EPA and DHA denote only the species of omega-3 oil and do not describe whether the components of such oils exist as, for example, triglycerides, diglycerides, monoglycerides, free acids, esters, or salts.

“HMG-CoA reductase inhibitor” as used herein includes, but is not limited to, pravastatin, fluvastatin, atorvastatin, lovastatin, simvastatin, rosuvastatin, and pitavastatin, cerivastatin. HMG-CoA reductase inhibitor s may be in the form of a salt, hydrate, solvate, polymorph, or a co-crystal. Statins may also be in the form of a hydrate, solvate, polymorph, or a co-crystal of a salt. Statins may also be present in the free acid or lactone form according to the present invention.

“Salt form of an HMG-CoA reductase inhibitor” as used herein is a salt of an HMG-CoA reductase inhibitor. Suitable salts include, but are not limited to, sodium, calcium, magnesium, potassium, and zinc. For example, the salt form of a specific HMG-CoA reductase inhibitor such as atorvastatin could be selected from atorvastatin calcium, atorvastatin sodium, atorvastatin potassium, atorvastatin magnesium, etc. Any pharmaceutically acceptable salt form could be used. Such salts may be present in form of a hydrate, solvate, polymorph, co-crystal or amorphous form.

### Compositions

In one embodiment, the invention is a pharmaceutical composition comprising a capsule containing an omega-3 oil, a salt form of an HMG-CoA reductase inhibitor, and a salt, wherein the salt form of HMG-CoA reductase inhibitor and the salt contain the same counterion. In another embodiment, the invention is a capsule containing an omega-3 oil, an HMG-CoA reductase inhibitor, and 0.05 to 1.5 mmol/ml of calcium. In a further embodiment, the invention is a capsule containing an omega-3 oil, an HMG-CoA reductase inhibitor, and calcium wherein the ratio of HMG-CoA reductaseinhibitor:calcium ranges from 10:0.05 to 10:1.5. In a still further embodiment, the invention is a capsule comprising an omega-3 oil, an HMG-CoA reductase inhibitor, and a salt selected from calcium phosphate, calcium carbonate, and calcium chloride.

In one embodiment, a pharmaceutical composition comprises an omega-3 oil, a salt form of an HMG-CoA reductase inhibitor, and a salt of an alkali metal or alkaline earth metal, wherein the salt form of HMG-CoA reductase inhibitor and the salt of an alkali metal or alkaline earth metal contain the same counterion. In one aspect of the embodiment, said omega-3 oil comprises eicosapentaenoic acid. In another aspect of the embodiment, the omega-3 oil comprises docosahexaenoic acid. In a further aspect of the embodiment, the omega-3 oil comprises eicosapentaenoic acid and docosahexaenoic acid. In one aspect of the embodiment, the omega-3 oil has a purity of at least 75% of eicosapentaenoic acid, docosahexaenoic acid or both eicosapentaenoic acid and docosahexaenoic acid. In another aspect of the embodiment, the omega-3 oil has a purity of at least 85% of eicosapentaenoic acid, docosahexaenoic acid or both eicosapentaenoic acid and docosahexaenoic acid. In a further aspect of the embodiment, the omega-3 oil has a purity of at least 90% of eicosapentaenoic acid, docosahexaenoic acid or both eicosapentaenoic acid and docosahexaenoic acid. In a still further aspect of the embodiment, the omega-3 oil has a purity of at least 95% of eicosapentaenoic acid, docosahexaenoic acid or both eicosapentaenoic acid and docosahexaenoic acid. In one aspect of the embodiment, the omega-3 oil is in the form of a triglyceride. In another aspect of the embodiment, the omega-3 oil is in the form of an ethyl ester. In a further aspect of the embodiment, the omega-3 oil is in the form of a free fatty acid. In one aspect of the embodiment, the omega-3 oil is in the form of a phospholipid. In one aspect of the embodiment, the salt form of an HMG-CoA reductase inhibitor is selected from a potassium salt, a calcium salt, a magnesium salt, zinc salt or a sodium salt. In one aspect of the embodiment, the salt form of an HMG-CoA reductase inhibitor is a calcium salt and said salt of an alkali metal or alkaline earth metal is a calcium salt. In another aspect of the embodiment, the salt of an alkali metal or alkaline earth metal is a calcium salt and is selected from calcium phosphate, calcium carbonate, calcium chloride, calcium acetate, calcium alginate, calcium hydroxide, calcium lactate, calcium silicate, calcium stearate, calcium sulfate, and calcium oxide. In one aspect of the embodiment, the salt of an alkali metal or alkaline earth metal is selected from calcium phosphate, calcium carbonate, and calcium chloride. In another aspect of the embodiment, salt of an alkali metal or alkaline earth metal is a magnesium salt and is selected from magnesium silicate, magnesium oxide, magnesium carbonate, and magnesium tri-silicate. In a further aspect of the embodiment, salt of an alkali metal or alkaline earth metal is a zinc salt and is selected from zinc acetate and zinc oxide. In a still further aspect of the embodiment, the counterion of said salt of an alkali metal or alkaline earth metal is present at a concentration of 0.05 to 1.5 mmol/ml of omega-3 oil. In an additional aspect of the embodiment, the counterion of said calcium is present at a concentration of 0.05 to 1.5 mmol/ml. In one aspect of the embodiment, the salt form of an HMG-CoA reductase inhibitor is a calcium salt, said salt of an alkali metal or alkaline earth metal is a calcium salt and said omega-3 oil has a purity of at least 90% of eicosapentaenoic acid, docosahexaenoic acid or both eicosapentaenoic acid and docosahexaenoic acid. In an additional aspect of the embodiment, the form of an HMG-CoA reductase inhibitor is atorvastatin calcium, said salt of an alkali metal or alkaline earth metal is a calcium salt and said omega-3 oil has a purity of at least 90% of eicosapentaenoic acid, docosahexaenoic acid or both eicosapentaenoic acid and docosahexaenoic acid. In a further aspect of the embodiment, the salt form of an HMG-CoA reductase inhibitor is atorvastatin calcium, said salt of an alkali metal or alkaline earth metal is a calcium salt, said omega-3 oil has a purity of at least 90% of eicosapentaenoic acid, docosahexaenoic acid or both eicosapentaenoic acid and docosahexaenoic acid and said omega-3 oil is in the form of an ethyl ester. In a still further aspect of the embodiment, the salt form of an HMG-CoA reductase inhibitor is atorvastatin calcium, said salt of an alkali metal or alkaline earth metal is a calcium salt, said omega-3 oil has a purity of at least 90% of eicosapentaenoic acid, docosahexaenoic acid or both eicosapentaenoic acid and docosahexaenoic acid and said omega-3 oil is in the form of a triglyceride. In one aspect of the embodiment, the salt form of an HMG-CoA reductase inhibitor is atorvastatin calcium, said salt of an alkali metal or alkaline earth metal is a calcium salt selected from calcium chloride, calcium phosphate, and calcium carbonate and said omega-3 oil has a purity of at least 90% of eicosapentaenoic acid, docosahexaenoic acid or both eicosapentaenoic acid and docosahexaenoic acid. In another aspect of the embodiment, the salt form of an HMG-CoA reductase inhibitor is atorvastatin calcium, said salt of an alkali metal or alkaline earth metal is calcium chloride, said omega-3 oil has a purity of at least 90% of eicosapentaenoic acid, docosahexaenoic acid or both eicosapentaenoic acid and docosahexaenoic acid and said omega-3 oil is in the form of an ethyl ester. In a further aspect of the embodiment, the salt form of an HMG-CoA reductase inhibitor is atorvastatin calcium, said salt of an alkali metal or alkaline earth metal is calcium chloride, said omega-3 oil has a purity of at least 90% of eicosapentaenoic acid, docosahexaenoic acid or both eicosapentaenoic acid and docosahexaenoic acid and said omega-3 oil is in the form of a triglyceride. In a still further aspect of the embodiment, the salt form of an HMG-CoA reductase inhibitor is rosuvastatin calcium, said salt of an alkali metal or alkaline earth metal is a calcium salt and said omega-3 oil has a purity of at least 90% of eicosapentaenoic acid, docosahexaenoic acid or both eicosapentaenoic acid and docosahexaenoic acid. In one aspect of the embodiment, the salt form of an HMG-CoA reductase inhibitor is rosuvastatin calcium, said salt of an alkali metal or alkaline earth metal is a calcium salt, said omega-3 oil has a purity of at least 90% of eicosapentaenoic acid, docosahexaenoic acid or both eicosapentaenoic acid and docosahexaenoic acid and said omega-3 oil is in the form of an ethyl ester. In another aspect of the embodiment, the salt form of an HMG-CoA reductase inhibitor is rosuvastatin calcium, said salt of an alkali metal or alkaline earth metal is a calcium salt, said omega-3 oil has a purity of at least 90% of eicosapentaenoic acid, docosahexaenoic acid or both eicosapentaenoic acid and docosahexaenoic acid and said omega-3 oil is in the form of a triglyceride. In a further aspect of the embodiment, salt form of an HMG-CoA reductase inhibitor is rosuvastatin calcium, said salt of an alkali metal or alkaline earth metal is a calcium salt, said omega-3 oil has a purity of at least 90% of eicosapentaenoic acid, docosahexaenoic acid or both eicosapentaenoic acid and docosahexaenoic acid and said omega-3 oil is in the form of a free fatty acid. In a still further aspect of the embodiment, salt form of an HMG-CoA reductase inhibitor is rosuvastatin calcium, said salt of an alkali metal or alkaline earth metal is a calcium salt selected from calcium chloride, calcium phosphate, and calcium carbonate and said omega-3 oil has a purity of at least 90% of eicosapentaenoic acid, docosahexaenoic acid or both eicosapentaenoic acid and docosahexaenoic acid. In one aspect of the embodiment, the salt form of an HMG-CoA reductase inhibitor is rosuvastatin calcium, said salt of an alkali metal or alkaline earth metal is calcium chloride, said omega-3 oil has a purity of at least 90% of eicosapentaenoic acid, docosahexaenoic acid or both eicosapentaenoic acid and docosahexaenoic acid and said omega-3 oil is in the form of an ethyl ester. In another aspect of the embodiment, salt form of an HMG-CoA reductase inhibitor is rosuvastatin calcium, said salt of an alkali metal or alkaline earth metal is calcium chloride, said omega-3 oil has a purity of at least 90% of eicosapentaenoic acid, docosahexaenoic acid or both eicosapentaenoic acid and docosahexaenoic acid and said omega-3 oil is in the form of a free fatty acid. In one aspect of the embodiment, the composition contains between 5-40 mg of said HMG-CoA reductase inhibitor.

In another embodiment, an oral dosage form comprises an omega-3 oil, an HMG-CoA reductase inhibitor, and 0.05 to 1.5 mmol/ml of calcium. In one aspect of the embodiment, the omega-3 oil comprises eicosapentaenoic acid. In another aspect of the embodiment, the omega-3 oil comprises docosahexaenoic acid. In a further aspect of the embodiment, the omega-3 oil comprises eicosapentaenoic acid and docosahexaenoic acid. In a still further aspect of the embodiment, the omega-3 oil has a purity of at least 75% of eicosapentaenoic acid, docosahexaenoic acid or both eicosapentaenoic acid and docosahexaenoic acid. In one aspect of the embodiment, the omega-3 oil has a purity of at least 85% of eicosapentaenoic acid, docosahexaenoic acid or both eicosapentaenoic acid and docosahexaenoic acid. In another aspect of the embodiment, the omega-3 oil has a purity of at least 90% of eicosapentaenoic acid, docosahexaenoic acid or both eicosapentaenoic acid and docosahexaenoic acid. In a further aspect of the embodiment, the omega-3 oil has a purity of at least 95% of eicosapentaenoic acid, docosahexaenoic acid or both eicosapentaenoic acid and docosahexaenoic acid. In one aspect of the embodiment, the omega-3 oil is in the form of a triglyceride. In another aspect of the embodiment, the omega-3 oil is in the form of an ethyl ester. In a further aspect of the embodiment, the omega-3 oil is in the form of a free fatty acid. In a still further aspect of the embodiment, the omega-3 oil is in the form of a phospholipid. In one aspect of the embodiment, the composition contains between 10-40 mg of said HMG-CoA reductase inhibitor. In another aspect of the embodiment, the HMG-CoA reductase inhibitor is selected from the list consisting of atorvastatin, rosuvastatin, pravastatin, pitavastatin, fluvastatin, and simvastatin. In a further aspect of the embodiment, the HMG-CoA reductase inhibitor is selected from the list consisting of atorvastatin calcium, rosuvastatin calcium, pravastatin calcium, pitavastatin calcium, and fluvastatin calcium.

In an additional embodiment, an oral dosage form comprises an omega-3 oil, an HMG-CoA reductase inhibitor, and calcium wherein the ratio of HMG-CoA reductase inhibitor:calcium ranges from 10:0.0.05 to 10:1.5. In one aspect of the embodiment, the omega-3 oil comprises eicosapentaenoic acid. In another aspect of the embodiment, the omega-3 oil comprises docosahexaenoic acid. In a further aspect of the embodiment, the omega-3 oil comprises eicosapentaenoic acid and docosahexaenoic acid. In a still further aspect of the embodiment, the omega-3 oil has a purity of at least 75% of eicosapentaenoic acid, docosahexaenoic acid or both eicosapentaenoic acid and docosahexaenoic acid. In one aspect of the embodiment, the omega-3 oil has a purity of at least 85% of eicosapentaenoic acid, docosahexaenoic acid or both eicosapentaenoic acid and docosahexaenoic acid. In another aspect of the embodiment, the omega-3 oil has a purity of at least 90% of eicosapentaenoic acid, docosahexaenoic acid or both eicosapentaenoic acid and docosahexaenoic acid. In a further aspect of the embodiment, omega-3 oil has a purity of at least 95% of eicosapentaenoic acid, docosahexaenoic acid or both eicosapentaenoic acid and docosahexaenoic acid. In a still further aspect of the embodiment, the omega-3 oil is in the form of a triglyceride. In one aspect of the embodiment, the omega-3 oil is in the form of an ethyl ester. In another aspect of the embodiment, the omega-3 oil is in the form of a free fatty acid. In a further aspect of the embodiment, the omega-3 oil is in the form of a phospholipid. In a still further aspect of the embodiment, the composition contains between 5-40 mg of said HMG-CoA reductase inhibitor. In one aspect of the embodiment, the HMG-CoA reductase inhibitor is selected from the list consisting of atorvastatin, rosuvastatin, pravastatin, pitavastatin, fluvastatin, and simvastatin. In another aspect of the embodiment, HMG-CoA reductase inhibitor is selected from the list consisting of atorvastatin calcium, rosuvastatin calcium, pravastatin calcium, pitavastatin calcium, and fluvastatin calcium.

In a further embodiment, an oral dosage form comprising an omega-3 oil, an HMG-CoA reductase inhibitor, and a salt selected from calcium phosphate, calcium carbonate, and calcium chloride. In one aspect of the embodiment, the omega-3 oil comprises eicosapentaenoic acid. In another aspect of the embodiment, the omega-3 oil comprises docosahexaenoic acid. In a further aspect of the embodiment, the omega-3 oil comprises eicosapentaenoic acid and docosahexaenoic acid. In one aspect of the embodiment, omega-3 oil has a purity of at least 75% of eicosapentaenoic acid, docosahexaenoic acid or both eicosapentaenoic acid and docosahexaenoic acid. In one aspect of the embodiment, the omega-3 oil has a purity of at least 85% of eicosapentaenoic acid, docosahexaenoic acid or both eicosapentaenoic acid and docosahexaenoic acid. In another aspect of the embodiment, omega-3 oil has a purity of at least 90% of eicosapentaenoic acid, docosahexaenoic acid or both eicosapentaenoic acid and docosahexaenoic acid. In a further aspect of the embodiment, the omega-3 oil has a purity of at least 95% of eicosapentaenoic acid, docosahexaenoic acid or both eicosapentaenoic acid and docosahexaenoic acid. In a still further aspect of the embodiment, the omega-3 oil is in the form of a triglyceride. In one aspect of the embodiment, the omega-3 oil is in the form of an ethyl ester. In another aspect of the embodiment, the omega-3 oil is in the form of a free fatty acid. In a further aspect of the embodiment, the omega-3 oil is in the form of a phospholipid. In a still further aspect of the embodiment, the composition contains between 10-40 mg of said HMG-CoA reductase inhibitor. In one aspect of the embodiment, the HMG-CoA reductase inhibitor is selected from the list consisting of atorvastatin, rosuvastatin, pravastatin, pitavastatin, fluvastatin, and simvastatin. In another aspect of the embodiment, the HMG-CoA reductase inhibitor is selected from the list consisting of atorvastatin calcium, rosuvastatin calcium, pravastatin calcium, pitavastatin calcium, and fluvastatin calcium. In a further aspect of the embodiment, the salt is sodium chloride.

In one embodiment, the HMG-CoA reductase inhibitor remains substantially suspended in the omega-3 oil after 24 hours at room temperature storage. In another embodiment, at least 80% of said HMG-CoA reductase inhibitor remains suspended in the omega-3 oil after 24 hours at room temperature storage. In an additional embodiment, at least 80% of said HMG-CoA reductase inhibitor remains suspended in the omega-3 oil after one month at room temperature storage.

In another embodiment, the invention comprises a method of stabilizing an HMG-CoA reductase inhibitor in an omega-3 oil comprising the steps of combining an HMG-CoA reductase inhibitor, an omega-3 oil, and salt of an alkali metal or alkaline earth metal. In another aspect of the embodiment, the HMG-CoA reductase inhibitor remains substantially suspended in the omega-3 oil after 24 hours at room temperature storage. In a further aspect of the embodiment, at least 80% of said HMG-CoA reductase inhibitor remains suspended in the omega-3 oil after 24 hours at room temperature storage. In an additional aspect of the embodiment, at least 80% of said HMG-CoA reductase inhibitor remains suspended in the omega-3 oil after one month at room temperature storage. In one aspect of the embodiment, the omega-3 oil comprises eicosapentaenoic acid. In another aspect of the embodiment, the omega-3 oil comprises docosahexaenoic acid. In a further aspect of the embodiment, the omega-3 oil comprises eicosapentaenoic acid and docosahexaenoic acid. In a still further aspect of the embodiment, the omega-3 oil has a purity of at least 75% of eicosapentaenoic acid, docosahexaenoic acid or both eicosapentaenoic acid and docosahexaenoic acid. In one aspect of the embodiment, the omega-3 oil has a purity of at least 85% of eicosapentaenoic acid, docosahexaenoic acid or both eicosapentaenoic acid and docosahexaenoic acid. In another aspect of the embodiment, the omega-3 oil has a purity of at least 90% of eicosapentaenoic acid, docosahexaenoic acid or both eicosapentaenoic acid and docosahexaenoic acid. In a further aspect of the embodiment, the omega-3 oil has a purity of at least 95% of eicosapentaenoic acid, docosahexaenoic acid or both eicosapentaenoic acid and docosahexaenoic acid. In a still further aspect of the embodiment, the omega-3 oil is in the form of a triglyceride. In one aspect of the embodiment, the omega-3 oil is in the form of an ethyl ester. In another aspect of the embodiment, the omega-3 oil is in the form of a free fatty acid. In a further aspect of the embodiment, the omega-3 oil is in the form of a phospholipid. In still further aspect of the embodiment, the method comprises suspending between about 5 mg and about 40 mg of HMG-CoA reductase inhibitor in said omega-3 oil.

In an additional embodiment, the invention comprises a suspension of salt form of an HMG-CoA reductase inhibitor in an omega-3 oil. In one embodiment, the suspension comprises solid crystalline particles of the salt form of an HMG-CoA reductase inhibitor in an omega-3 oil. In another embodiment, the suspension comprises solid amorphous particles of the salt form of an HMG-CoA reductase inhibitor in an omega-3 oil. Also included in the present invention are pharmaceutical formulations comprising suspensions of the salt form of an HMG-CoA reductase inhibitor in an omega-3 oil where a portion of said one or more the salt form of an HMG-CoA reductase inhibitor is solubilized in the omega-3 oil or in additional component(s) of the formulation. For example, in another embodiment, the present invention provides a pharmaceutical formulation comprising an omega-3 oil and the salt form of an HMG-CoA reductase inhibitor, wherein at least about 1.00, 2.00, 3.00, 4.00, 5.00, 6.00, 7.00, 8.00, 9.00, 10.00, 15.00, 20.00, 30.00, 40.00, or 50.00 percent statin(s) by weight is/are in solution while the remaining statin(s) is/are present in suspension.

In one embodiment, the invention comprises 1 gram of omega-3 oil and 5-50 mg of HMG-CoA reductase inhibitor. In an additional embodiment, the invention comprises 1 gram of omega-3 oil and 5 mg of HMG-CoA reductase inhibitor. In another embodiment, the invention comprises 1 gram of omega-3 oil and 10 mg of HMG-CoA reductase inhibitor. In an additional embodiment, the invention comprises 1 gram of omega-3 oil and 15 mg of HMG-CoA reductase inhibitor. In a further embodiment, the invention comprises 1 gram of omega-3 oil and 20 mg of HMG-CoA reductase inhibitor. In a still further embodiment, the invention comprises 1 gram of omega-3 oil and 30 mg of HMG-CoA reductase inhibitor. In one embodiment, the invention comprises 1 gram of omega-3 oil and 40 mg of HMG-CoA reductase inhibitor. In another embodiment, the invention comprises 1 gram of omega-3 oil and 50 mg of HMG-CoA reductase inhibitor.

In one embodiment, the invention comprises 1 gram of omega-3 oil and 5-50 mg of atorvastatin. In an additional embodiment, the invention comprises 1 gram of omega-3 oil and 5 mg of atorvastatin. In another embodiment, the invention comprises 1 gram of omega-3 oil and 10 mg of atorvastatin. In an additional embodiment, the invention comprises 1 gram of omega-3 oil and 15 mg of atorvastatin. In a further embodiment, the invention comprises 1 gram of omega-3 oil and 20 mg of atorvastatin. In a still further embodiment, the invention comprises 1 gram of omega-3 oil and 30 mg of atorvastatin. In one embodiment, the invention comprises 1 gram of omega-3 oil and 40 mg of atorvastatin. In another embodiment, the invention comprises 1 gram of omega-3 oil and 50 mg of atorvastatin.

In one embodiment, the invention comprises 1 gram of omega-3 oil and 5-50 mg of rosuvastatin. In an additional embodiment, the invention comprises 1 gram of omega-3 oil and 5 mg of rosuvastatin. In another embodiment, the invention comprises 1 gram of omega-3 oil and 10 mg of rosuvastatin. In an additional embodiment, the invention comprises 1 gram of omega-3 oil and 15 mg of rosuvastatin. In a further embodiment, the invention comprises 1 gram of omega-3 oil and 20 mg of rosuvastatin. In a still further embodiment, the invention comprises 1 gram of omega-3 oil and 30 mg of rosuvastatin. In one embodiment, the invention comprises 1 gram of omega-3 oil and 400 mg of rosuvastatin. In another embodiment, the invention comprises 1 gram of omega-3 oil and 5-50 mg of rosuvastatin.

In one embodiment, the invention comprises 800-1200 mg of omega-3 oil and 2.5-50 mg of HMG-CoA reductase inhibitor. In an additional embodiment, the invention comprises 800-1200 mg of omega-3 oil and 5 mg of HMG-CoA reductase inhibitor. In another embodiment, the invention comprises 800-1200 mg of omega-3 oil and 10 mg of HMG-CoA reductase inhibitor. In an additional embodiment, the invention comprises 800-1200 mg of omega-3 oil and 15 mg of HMG-CoA reductase inhibitor. In a further embodiment, the invention comprises 800-1200 mg of omega-3 oil and 20 mg of HMG-CoA reductase inhibitor. In a still further embodiment, the invention comprises 800-1200 mg of omega-3 oil and 30 mg of HMG-CoA reductase inhibitor. In one embodiment, the invention comprises 800-1200 mg of omega-3 oil and 40 mg of HMG-CoA reductase inhibitor. In another embodiment, the invention comprises 800-1200 mg of omega-3 oil and 50 mg of HMG-CoA reductase inhibitor.

In one embodiment, the invention comprises 800-1200 mg of omega-3 oil and 2.5-50 mg of atorvastatin. In an additional embodiment, the invention comprises 800-1200 mg of omega-3 oil and 5 mg of atorvastatin. In another embodiment, the invention comprises 800-1200 mg of omega-3 oil and 10 mg of atorvastatin. In an additional embodiment, the invention comprises 800-1200 mg of omega-3 oil and 15 mg of atorvastatin. In a further embodiment, the invention comprises 800-1200 mg of omega-3 oil and 20 mg of atorvastatin. In a still further embodiment, the invention comprises 800-1200 mg of omega-3 oil and 30 mg of atorvastatin. In one embodiment, the invention comprises 800-1200 mg of omega-3 oil and 40 mg of atorvastatin. In another embodiment, the invention comprises 800-1200 mg of omega-3 oil and 50 mg of atorvastatin.

In one embodiment, the invention comprises 800-1200 mg of omega-3 oil and 2.5-50 mg of rosuvastatin. In an additional embodiment, the invention comprises 800-1200 mg of omega-3 oil and 5 mg of rosuvastatin. In another embodiment, the invention comprises 800-1200 mg of omega-3 oil and 10 mg of rosuvastatin. In an additional embodiment, the invention comprises 800-1200 mg of omega-3 oil and 15 mg of rosuvastatin. In a further embodiment, the invention comprises 800-1200 mg of omega-3 oil and 20 mg of rosuvastatin. In a still further embodiment, the invention comprises 800-1200 mg of omega-3 oil and 30 mg of rosuvastatin. In one embodiment, the invention comprises 800-1200 mg of omega-3 oil and 400 mg of rosuvastatin. In another embodiment, the invention comprises 800-1200 mg of omega-3 oil and 2.5-50 mg of rosuvastatin.

### Omega-3 Oil

In one embodiment, the purity of omega-3 oil is at least about 50 percent by weight, at least about 60 percent by weight, at least about 70 percent by weight, at least about 75 percent by weight, at least about 80 percent by weight, or at least about 85 percent by weight. In another embodiment, the purity of omega-3 esters or omega-3 oil is about 25, 30, 35, 40, 45, 50, 55, 60, 65, 70, 75, 80, 85, 90, 95, 99 percent or more by weight. In another embodiment, the purity of omega-3 oil is between about 25 and about 100 percent by weight, between about 40 and about 100 percent by weight, between about 50 and about 100 percent by weight, between about 60 and about 100 percent by weight, between about 70 and about 100 percent by weight, between about 75 and about 100 percent by weight, between about 75 and about 95 percent by weight, between about 75 and about 90 percent by weight, or between about 80 and about 85 percent by weight. In another embodiment, the purity of omega-3 oil is about 100 percent by weight, about 99 percent by weight, about 96 percent by weight, about 92 percent by weight, about 90 percent by weight, about 85 percent by weight, about 80 percent by weight, about 75 percent by weight, about 70 percent by weight, about 65 percent by weight, about 60 percent by weight, about 55 percent by weight, or about 50 percent by weight.

In another embodiment, the omega-3 oil comprises at least 95% by weight EPA. In another embodiment, the omega-3 oil comprises at least 95% by weight EPA, about 0.2% to about 0.5% by weight ethyl octadecatetraenoate, about 0.05% to about 0.25% by weight ethyl nonadecapentaenoate, about 0.2% to about 0.45% by weight ethyl arachidonate, about 0.3% to about 0.5% by weight ethyl eicosatetraenoate and about 0.05% to about 0.32% ethyl heneicosapentaenoate. In another embodiment, omega-3 oil contains substantially no amount of docosahexaenoic acid.

In one embodiment, the omega-3 oil comprises EPA and DHA which are present in relative amounts of 1:2 to 2:1, and constitute at least 75% of the total fatty acids. In another embodiment, the invention comprises at least 90% by weight of long chain, polyunsaturated omega-3 fatty acids of which EPA and DHA constitute at least 85% by weight of the total fatty acids and are present in a ratio of EPA:DHA from 1:1 to 2:1 especially about 3:2.

In a still further embodiment, the invention comprises at least 75% eicosapentaenoic acid and docosahexaenoic acid by weight of the fatty acid oil mixture, wherein the EPA and DHA are in free acid form. In an additional embodiment, the omega-3 oil comprises eicosapentaenoic acid (EPA), docosahexaenoic acid (DHA), and docosapentaenoic acid (DPA) wherein the weight ratio of EPA:DHA is in the range of 5.7:1-6.3:1, the formulation contains about 90% or greater by weight omega-3 oil, and the EPA, DHA and DPA comprise about 82% by weight of the content of the formulation.

In one embodiment, a pharmaceutical composition comprises about 1 gram of omega-3 oil. In an additional embodiment, a pharmaceutical composition comprises about 2 grams of omega-3 oil. In another embodiment, a pharmaceutical composition comprises about 3 grams of omega-3 oil. In a further embodiment, a pharmaceutical composition comprises about 4 grams of omega-3 oil. In a still further embodiment, a pharmaceutical composition comprises about 800 mg of omega-3 oil. In one embodiment, a pharmaceutical composition comprises about 900 mg of omega-3 oil. In another embodiment, a pharmaceutical composition comprises about 950 mg of omega-3 oil. In one embodiment, a pharmaceutical composition comprises about 800-1200 mg of omega-3 oil. In another embodiment, a pharmaceutical composition comprises about 800-1200 mg of omega-3 oil per capsule.

### Salts

In one embodiment, the invention comprises a salt of an alkali metal or alkaline earth metal. In one embodiment, the salt of an alkali metal or alkaline earth metal of this invention selected from a potassium salt, a calcium salt, a magnesium salt, zinc salt or a sodium salt. In another embodiment, the salt of an alkali metal or alkaline earth metal is a calcium salt. In an additional embodiment, the calcium salt is selected from calcium phosphate, calcium carbonate, calcium chloride, calcium acetate, calcium alginate, calcium hydroxide, calcium lactate, calcium silicate, calcium stearate, calcium sulfate, and calcium oxide. In a still further embodiment, calcium salt is selected from calcium phosphate, calcium carbonate, and calcium chloride. In another embodiment, the salt of an alkali metal or alkaline earth metal is a magnesium salt. In an additional embodiment, the magnesium salt is selected from magnesium silicate, magnesium oxide, magnesium carbonate, and magnesium tri-silicate. In another embodiment, the salt of an alkali metal or alkaline earth metal is a zinc salt. In a still further embodiment, zinc salt is selected from zinc acetate and zinc oxide. In a still further embodiment, calcium salt is selected from calcium phosphate, calcium carbonate, and calcium chloride.

In another embodiment, the counterion of said salt of an alkali metal or alkaline earth metal is present at a concentration of 0.05 to 1.5 mmol/ml of omega-3 oil. In a still further embodiment, the counterion of said calcium salt is present at a concentration of 0.07 to 1.2 mmol/ml. In another embodiment, the counterion of said salt of an alkali metal or alkaline earth metal is present at a concentration of 0.07 to 1.2 mmol/ml of omega-3 oil. In another embodiment, the counterion of said salt of an alkali metal or alkaline earth metal is present at a concentration of 0.07 to 1.0 mmol/ml of omega-3 oil.

### Capsules

In one embodiment, compositions of this invention are contained in a capsule. In another embodiment, compositions of this invention are contained in a gelatin capsule. In another embodiment, compositions of this invention are contained in a hard gelatin capsule sealed with an appropriate technique. In another embodiment of the present invention, there is a soft gelatin capsule as defined above, wherein the omega-3 fatty acids comprise eicosapentaenoic acid (EPA), docosahexaenoic acid (DHA), or a mixture thereof.

In one embodiment, a pharmaceutical composition comprises one capsule. In another embodiment, a pharmaceutical composition comprises two capsules. In an additional embodiment, a pharmaceutical composition comprises three capsules. In a further embodiment, a pharmaceutical composition comprises four capsules. In one embodiment, the capsule size is selected from 000, 00, 0, 1, 2, and 3 and any shape variants thereof.

The shape and size of the soft gelatin capsules can vary in accordance with the invention. The shape of the capsule may be, but is not limited to, round, oval, oblong, or a non-standard shape. Typical soft gelatin dosage form shapes and sizes may be, but are not limited to, those as shown in Table 1. The invention provides the capability to incorporate into the soft gelatin capsule a wide range of solid dosage components (form and shape) to manufacture an infinite variety of soft gelatin capsule shapes and sizes.

TABLE 1
Nominal Soft Gelatin Capsule Shapes and Sizes

| | Oblong Shape | | Oval Shape | |
|---|---|---|---|---|
| Nominal | Minimum | Maximum | Minimum | Maximum |
| 1 | 0.03 | 0.08 | 0.03 | 0.06 |
| 2 | 0.08 | 0.14 | 0.06 | 0.09 |
| 3 | 0.14 | 0.20 | 0.11 | 0.17 |
| 4 | 0.20 | 0.30 | 0.15 | 0.22 |
| 5 | 0.26 | 0.37 | 0.23 | 0.30 |
| 6 | 0.32 | 0.46 | 0.26 | 0.38 |
| 8 | 0.43 | 0.63 | 0.33 | 0.48 |
| 10 | 0.53 | 0.76 | 0.42 | 0.60 |
| 12 | 0.64 | 0.93 | 0.50 | 0.73 |
| 14 | 0.75 | 1.06 | 0.60 | 0.85 |
| 16 | 0.85 | 1.23 | 0.70 | 1.00 |
| 18 | 0.96 | 1.40 | 0.76 | 1.10 |
| 20 | 1.10 | 1.55 | 0.85 | 1.20 |
| 22 | 1.20 | 1.70 | 0.95 | 1.35 |
| 24 | 1.30 | 1.85 | 1.05 | 1.46 |
| 26 | 1.40 | 2.00 | 1.13 | 1.60 |
| 28 | 1.50 | 2.15 | 1.23 | 1.70 |
| 30 | 1.65 | 2.30 | 1.30 | 1.85 |

The shell of the soft gelatin capsule may be formed from plasticized gelatin or other functional polymeric materials that are typically used for encapsulation of liquids, fluids, pastes or other fill materials. The outer shell of the soft gelatin capsule may be coated with one or more coatings, including but not limited to, immediate release coatings, protective coatings, enteric or delayed release coatings, sustained release coating, barrier coatings, and combinations thereof. The one or more coatings on the outer shell of the soft gelatin capsule may be useful to provide controlled release of the soft gelatin capsule, protect the soft gelatin shell from degradation, or deliver one or more active ingredients which may be the same or different as those in the liquid phase and solid dosage form. Alternatively, additives such as pectin or synthetic polymers may be incorporated into the soft gelatin capsule shell to slow the dissolution on ingestion. Such coatings or additives to the soft gelatin shell phase are well described in the literature and known to those experts in the field. The one or more coatings outer shell of the soft gelatin capsule may be applied by any conventional technique, including but not limited to, pan coating, fluid bed coating or spray coating.

In other embodiments, the invention provides a pharmaceutical composition comprising an omega-3 oil encapsulated in a capsule shell comprising a film forming material and a hygroscopic plasticizer, wherein the weight ratio of film-forming material to hygroscopic plasticizer is not less than about 2.5:1. Further, the capsule shell can optionally comprise a non-hygroscopic plasticizer. In one embodiment, the capsule contains no chemically modified gelatin, for example succinated or succinylated gelatin.

In another embodiment, a soft gelatin capsule contains a pharmaceutical formulation comprising at least one omega-3 oil in free acid form characterized in that the capsule comprises gelatin extracted by an extraction process comprising acid pre-treatment of a collagen source.

In a further embodiment, a pharmaceutical composition comprises a capsule containing as an HMG-CoA reductase inhibitor and an omega-3 polyunsaturated acid in free acid form or a pharmaceutically acceptable salt thereof, characterized in that the coating of the capsule is of a material which dissolves in a time but not pH dependent manner and is resistant to the release of the omega-3 polyunsaturated acid for a period of 30 to 60 minutes at pH 5.5 such that said omega-3 polyunsaturated acid is released in the small intestine.

### Methods of Treatment

In one embodiment, the invention comprises treating a patient with an HMG-CoA reductase inhibitor and an omega-3 oil at the same time, wherein both products are co-formulated together. In one aspect of the invention, a patient is treated for both high cholesterol and high triglycerides. In another embodiment, the invention is used as an adjunct to diet to reduce elevated total-C, LDL-C, apo B, and TG levels and to increase HDL-C in patients with primary hypercholesterolemia (heterozygous familial and nonfamilial) and mixed dyslipidemia (Fredrickson Types IIa and IIb). In a further embodiment, the invention is used as an adjunct to diet for the treatment of patients with elevated serum TG levels (Fredrickson Type IV). In a still further embodiment, the invention is used as or the treatment of patients with primary dysbetalipoproteinemia (Fredrickson Type III) who do not respond adequately to diet. In one embodiment, the invention is used as to reduce total-C and LDL-C in patients with homozygous familial hypercholesterolemia as an adjunct to other lipid-lowering treatments (e.g., LDL apheresis) or if such treatments are unavailable. In another embodiment, the invention is used as an adjunct to diet to reduce total-C, LDL-C, and apo B levels in boys and postmenarchal girls, 10 to 17 years of age, with heterozygous familial hypercholesterolemia if after an adequate trial of diet therapy the following findings are present: LDL-C remains≧190 mg/dL or LDL-C remains≧160 mg/dL and there is a positive family history of premature cardiovascular disease or two or more other CVD risk factors are present in the pediatric patient.

In one embodiment, the invention comprises treating a patient with an HMG-CoA reductase inhibitor and an omega-3 oil at the same time, wherein both products are co-formulated together. In one aspect of the invention, a patient is treated for both high cholesterol and high triglycerides. In another embodiment, the invention is used as an adjunct to diet to reduce elevated total-C, LDL-C, apo B, and TG levels and to increase HDL-C in patients with primary hypercholesterolemia (heterozygous familial and nonfamilial) and mixed dyslipidemia (Fredrickson Types IIa and IIb) while also providing a patient with 1-4 grams of omega-3. In a further embodiment, the invention is used as an adjunct to diet for the treatment of patients with elevated serum TG levels (Fredrickson Type IV) while also providing a patient with 1-4 grams of omega-3. In a still further embodiment, the invention is used as or the treatment of patients with primary dys-betalipoproteinemia (Fredrickson Type III) who do not respond adequately to diet. In one embodiment, the invention is used as to reduce total-C and LDL-C in patients with homozygous familial hypercholesterolemia as an adjunct to other lipid-lowering treatments (e.g., LDL apheresis) while also providing a patient with 1-4 grams of omega-3. In another embodiment, the invention is used as an adjunct to diet to reduce total-C, LDL-C, and apo B levels in boys and postmenarchal girls, 10 to 17 years of age, with heterozygous familial hypercholesterolemia if after an adequate trial of diet therapy the following findings are present: LDL-C remains≧190 mg/dL or LDL-C remains≧160 mg/dL and there is a positive family history of premature cardiovascular disease or two or more other CVD risk factors are present in the pediatric patient while also providing a patient with 1-4 grams of omega-3.

In various embodiments, the present invention provides pharmaceutical compositions and methods of using such compositions to treat and/or prevent cardiovascular-related diseases.

In one embodiment, the subject is on concomitant statin therapy. In another embodiment, the subject on statin therapy has a baseline fasting serum triglyceride level of about 200 mg/dL to about 500 mg/dL.

In one embodiment, the invention provides a method of lowering triglycerides in a subject on stable statin therapy having baseline fasting triglycerides of about 200 mg/dl to about 500 mg/dl, the method comprising administering to the subject a composition of this invention, wherein upon administering the composition to the subject daily for a period of 12 weeks the subject exhibits at least 5% lower fasting triglycerides than a control subject maintained on stable statin therapy. In another embodiment, upon administering the composition to the subject daily for a period of 12 weeks the subject exhibits no serum LDL-C increase, no statistically significant serum LDL-C increase, a serum LDL-C decrease, or the subject is statistically non-inferior to the control subjects (statin plus optional placebo) in regard to serum LDL-C elevation).

### EXAMPLES

### Example 1

Atorvastatin Ca (At) in ethyl ester of EPA (97% purity from Equatec) was prepared as follows: The drug substance was weighed into a glass vessel containing a magnetic stir bar and the oil was then added by volume to make a 10 mg/ml suspension. The clear oil made an off-white suspension with the drug, which was uniformly dispersed by magnetic stirring. A further addition of additives was then performed, as indicated in Table 2. Each formulation also had 1% BHA via ethanol solution (1% of volume of the final suspension from a 100 mg/mL solution of BHA in anhydrous ethanol, resulting in 1 mg/mL BHA or 0.1% w/V). While stirring the suspensions were individually transferred by mass into 250 ul glass vial inserts to about 260 mg, which was essentially a complete fill. Vials were crimped with a teflon-backed seal and placed on stability stations: 25 C/60% RH, 40 C/75% RH and −20 C for reference. Samples were pulled at monthly intervals and prepared for HPLC analysis using a C18 column, samples were transferred, fully dissolved and diluted to 10.0 ml with dry DMF. At and corresponding lactone were quantified from HPLC chromatograms against standards of the At API. Integration was done using the Perkin Elmer software supplied with the HPLC system.

TABLE 2
Additives in suspension or statin calcium salts in omega-3 oils

| Additive | Chemical | mg/mL | mmolCa | Ca from |
|---|---|---|---|---|
| (identifier) | formula | in oil | added/mL | statins* |
| Calcium | CaCO3 | 10 | 0.10 | 0.0093 |
| Carbonate (C) | | | | |
| Calcium | CaCl2 | 10 | 0.090 | 0.0093 |
| Chloride (K) | | | | |
| Calcium | CaHPO4 | 10 | 0.071 | 0.0093 |
| Phosphate (P) | | | | |

*assuming 20 mg/mL drug and an average molecular weight of 1075 for the two drugs.

### Example 2

Atorvastatin Ca (At) in omega-3 free fatty acid was prepared as follows: The drug substance was weighed into a glass vessel containing a magnetic stir bar and the oil was then added by volume to make a 10 mg/ml suspension. The clear oil made an off-white suspension with the drug, which was uniformly dispersed by magnetic stirring. A further addition of additives was then performed, as indicated in Table 1. Each formulation also had 1% BHA via ethanol solution (1% of volume of the final suspension from a 100 mg/mL solution of BHA in anhydrous ethanol, resulting in 1 mg/mL BHA or 0.1% w/V). While stirring the suspensions were individually transferred by mass into 250 ul glass vial inserts to about 260 mg, which was essentially a complete fill. Vials were crimped with a teflon-backed seal and placed on stability stations: 25 C/60% RH, 40 C/75% RH and −20 C for reference. Samples were pulled at monthly intervals and prepared for HPLC analysis using a C18 column, samples were transferred, fully dissolved and diluted to 10.0 ml with dry DMF. At and corresponding lactone were quantified from HPLC chromatograms against standards of the At API. Integration was done using the Perkin Elmer software supplied with the HPLC system.

TABLE 3
Additives in suspension or statin calcium salts in omega-3 oils

| Additive | Chemical | mg/mL | mmolCa | Ca from |
|---|---|---|---|---|
| (identifier) | formula | in oil | added/mL | statins* |
| Calcium | CaCO3 | 10 | 0.10 | 0.0093 |
| Carbonate (C) | | | | |
| Calcium | CaCl2 | 10 | 0.090 | 0.0093 |
| Chloride (K) | | | | |
| Calcium | CaHPO4 | 10 | 0.071 | 0.0093 |
| Phosphate (P) | | | | |

*assuming 20 mg/mL drug and an average molecular weight of 1075 for the two drugs.

### Example 3

Rosuvastatin Ca (Ro) in ethyl ester of EPA (97% purity from Equatec) was prepared as follows: The drug substance was weighed into a glass vessel containing a magnetic stir bar and the oil was then added by volume to make a 10 mg/ml suspension. The clear oil made an off-white suspension with the drug, which was uniformly dispersed by magnetic stirring. A further addition of additives was then performed, as indicated in Table 1. Each formulation also had 1% BHA via ethanol solution (1% of volume of the final suspension from a 100 mg/mL solution of BHA in anhydrous ethanol, resulting in 1 mg/mL BHA or 0.1% w/V). While stirring the suspensions were individually transferred by mass into 250 ul glass vial inserts to about 260 mg, which was essentially a complete fill. Vials were crimped with a teflon-backed seal and placed on stability stations: 25 C/60% RH, 40 C/75% RH and −20 C for reference. Samples were pulled at monthly intervals and prepared for HPLC analysis using a C18 column, samples were transferred, fully dissolved and diluted to 10.0 ml with dry DMF. At and corresponding lactone were quantified from HPLC chromatograms against standards of the At API. Integration was done using the Perkin Elmer software supplied with the HPLC system.

### Example 4

RosuvastatinCa (Ro) in omega-3 free fatty acid was prepared as follows: The drug substance was weighed into a glass vessel containing a magnetic stir bar and the oil was then added by volume to make a 10 mg/ml suspension. The clear oil made an off-white suspension with the drug, which was uniformly dispersed by magnetic stirring. A further addition of additives was then performed, as indicated in Table 4. Each formulation also had 1% BHA via ethanol solution (1% of volume of the final suspension from a 100 mg/mL solution of BHA in anhydrous ethanol, resulting in 1 mg/mL BHA or 0.1% w/V). While stirring the suspensions were individually transferred by mass into 250 ul glass vial inserts to about 260 mg, which was essentially a complete fill. Vials were crimped with a teflon-backed seal and placed on stability stations: 25 C/60% RH, 40 C/75% RH and −20 C for reference. Samples were pulled at monthly intervals and prepared for HPLC analysis using a C18 column, samples were transferred, fully dissolved and diluted to 10.0 ml with dry DMF. At and corresponding lactone were quantified from HPLC chromatograms against standards of the At API. Integration was done using the Perkin Elmer software supplied with the HPLC system.

TABLE 4
Additives in suspension or statin calcium salts in omega-3 oils

| Additive | Chemical | mg/mL in | mmolCa | Ca from |
|---|---|---|---|---|
| (identifier) | formula | oil | added/mL | statins* |
| Calcium | CaCO3 | 10 | 0.10 | 0.0093 |
| Carbonate (C) | | | | |
| Calcium | CaCl2 | 10 | 0.090 | 0.0093 |
| Chloride (K) | | | | |
| Calcium | CaHPO4 | 10 | 0.071 | 0.0093 |
| Phosphate (P) | | | | |

*assuming 20 mg/mL drug, 1/2 equivalent Ca and an average molecular weight of 1075 for the two drugs.

TABLE 5
Stability results for Ca salts of statins in three omega-3 oils at 40° C./75% RH after 1 month.

| Omega-3 oil | Additive | Atorvastatin | At Lactone | Rosuvastatin | Ro Lactone |
|---|---|---|---|---|---|
| Free acid | None | 22.8% | 75.6% | 39.1% | 60.0% |
| | CaCO3 | 101.8% | 1.5% | 105.4% | 0.25% |
| | CaCl2 | 87.3% | 13.5% | 105.2% | 0.28% |
| | CaHPO4 | 99.5% | 13.6% | 99.5% | 0.17% |
| Ethyl ester | None | 98.0% | 0.18% | 95.0% | 0.92% |
| | CaCO3 | 97.6% | 0.34% | 79.8% | 0.79% |
| | CaCl2 | 103.2% | 0.06% | 93.1% | 0.09% |
| | CaHPO4 | 100.9% | 0.21% | 96.2% | nd |
| Triglyceride | None | 85.3% | 4.6% | 100.6% | 2.5% |

TABLE 6
Stability of Atorvastatin in two omega-3 oils at 40° C./75%
RH and 25° C./60% RH after 3 months.

| | | 25° C./60% RH | | 40° C./75% RH | |
|---|---|---|---|---|---|
| Omega-3 | | | At | | At |
| oil | Additive | Atorvastatin | Lactone | Atorvastatin | Lactone |
| Free acid* | None | n/a | Tbd | Nov 23 | 3-month |
| | | | | | point |
| | CaCO3 | 77.3% | 24.6% | 76.4% | 35.2% |
| | CaCl2 | 84.2% | 18.6% | 90.7% | 19.8% |
| | CaHPO4 | 80.9% | 32.2% | 92.0% | 25.2% |
| Ethyl ester | None | n/a | Tbd | Nov 23 | 3-month |
| | | | | | point |
| | CaCO3 | 110.7% | 0.26% | 111.8% | 1.87% |
| | CaCl2 | 98.2% | 0.35% | 97.1% | 0.48% |
| | CaHPO4 | 98.0% | 0.38% | 90.8% | 4.80% |

*1.5-3% lactone was observed in the −20° C. reference samples in Free acid

TABLE 7
Stability of Rosuvastatin in two omega-3 oils at 40° C./75 %
RH and 25° C./60 % RH after 3 months.

| | | 25° C./60% RH | | 40° C./75% RH | |
|---|---|---|---|---|---|
| Omega-3 | | | Ro | | Ro |
| oil | Additive | Rosuvastatin | Lactone | Rosuvastatin | Lactone |
| Free acid | CaCO3 | 100.2% | 1.0% | 92.4% | 0.83% |
| | CaCl2 | 101.9% | 0.44% | 98.7% | 0.39% |
| | CaHPO4 | 99.9% | 0.74% | 102.2% | 0.69% |
| Ethyl ester | None | n/a | Tbd | Nov 23 | 3 month |
| | | | | | point |
| | CaCO3 | 101.9% | 0.31% | 86.1% | 5.56% |
| | CaCl2 | 110.4% | 0.23% | 108.8% | 1.03% |
| | CaHPO4 | 98.2% | 0.26% | 83.7% | 9.75% |

While this invention has been particularly shown and described with references to preferred embodiments thereof, it will be understood by those skilled in the art that various changes in form and details may be made therein without departing from the scope of the invention encompassed by the appended claims.

1. A pharmaceutical composition comprising an omega-3 oil, a salt form of an HMG-CoA reductase inhibitor, and a salt of an alkali metal or alkaline earth metal, wherein the salt form of HMG-CoA reductase inhibitor and the salt of an alkali metal or alkaline earth metal contain the same counterion.

2. The pharmaceutical composition of claim 1, wherein said omega-3 oil has a purity of at least 75% of eicosapentaenoic acid, docosahexaenoic acid or both eicosapentaenoic acid and docosahexaenoic acid.

3. The pharmaceutical composition of claim 1, wherein said omega-3 oil is in the form of an ethyl ester.

4. The pharmaceutical composition of claim 1, wherein said salt of an alkali metal or alkaline earth metal is a calcium salt and is selected from calcium phosphate, calcium carbonate, calcium chloride, calcium acetate, calcium alginate, calcium hydroxide, calcium lactate, calcium silicate, calcium stearate, calcium sulfate, and calcium oxide.

5. The pharmaceutical composition of claim 1, wherein said salt of an alkali metal or alkaline earth metal is a magnesium salt and is selected from magnesium silicate, magnesium oxide, magnesium carbonate, and magnesium tri-silicate.

6. The pharmaceutical composition of claim 1, wherein said counterion of said salt of an alkali metal or alkaline earth metal is present at a concentration of 0.05 to 1.5 mmol/ml of omega-3 oil.

7. The pharmaceutical composition of claim 1, wherein said composition contains between 10-40 mg of said HMG-CoA reductase inhibitor.

8. The pharmaceutical composition of claim 1, wherein said composition is in the form of a capsule.

9. An oral dosage form containing an omega-3 oil, an HMG-CoA reductase inhibitor, and 0.05 to 1.5 mmol/ml of calcium.

10. The pharmaceutical composition of claim 9, wherein said omega-3 oil has a purity of at least 75% of eicosapentaenoic acid, docosahexaenoic acid or both eicosapentaenoic acid and docosahexaenoic acid.

11. The pharmaceutical composition of claim 9, wherein said HMG-CoA reductase inhibitor is selected from the list consisting of atorvastatin calcium, rosuvastatin calcium, pravastatin calcium, pitavastatin calcium, and fluvastatin calcium.

12. A method of stabilizing an HMG-CoA reductase inhibitor in an omega-3 oil comprising the steps of:
combining an HMG-CoA reductase inhibitor, an omega-3 oil, and salt of an alkali metal or alkaline earth metal.

13. The method of claim 12, wherein the HMG-CoA reductase inhibitor remains substantially suspended in the omega-3 oil after 24 hours at room temperature storage.

14. The method of claim 12, wherein said omega-3 oil has a purity of at least 75% of eicosapentaenoic acid, docosahexaenoic acid or both eicosapentaenoic acid and docosahexaenoic acid.

15. The method of claim 12, wherein said omega-3 oil is in the form of a triglyceride.

16. The method of claim 12, wherein said omega-3 oil is in the form of an ethyl ester.

17. The method of claim 12, wherein said omega-3 oil is in the form of a free fatty acid.

18. The method of claim 12, wherein said method comprises suspending between about 2.5 mg and about 40 mg of HMG-CoA reductase inhibitor in said omega-3 oil.
